(19) United States
(12) Patent Application Publication
(10) Pub. No.: US 2009/0043195 A1
(43) Pub. Date: Feb. 12, 2009

(54) Ultrasound Touchscreen User Interface and Display

(75) Inventor: McKee D. Poland, Andover, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/577,025

(22) PCT Filed: Sep. 22, 2005
(86) PCT No.: PCT/IB2005/053142

§ 371 Date: Feb. 15, 2008

Related U.S. Application Data
(60) Provisional application No. 60/618,011, filed on Oct. 12, 2004.

(51) Int. Cl. A61B 8/00

(52) U.S. Cl. 600/437

(57) ABSTRACT

User interface for providing user control over device functions of an ultrasound imaging system (10) includes a touchscreen (18) and activation areas (22, 24, 26) defined thereon simultaneous images. Each activation area (22, 24, 26) has a unique assigned function relating to processing of the ultrasound images with an indication of the function being displayed on the activation area (22, 24, 26). A processor (16) is coupled to the touchscreen (18) for detecting a touch on the activation areas (22, 24, 26) and performing the function associated with each activation area (22, 24, 26) upon being touched. In this manner, all UI controls can be implemented as virtual controls by assigning the function of each control to an activation area (22, 24, 26) so that the user can simply touch the activation area and effect the desired control.

The present invention relates generally to medical diagnostic imaging systems, such as ultrasound imaging systems, and more particularly to a touchscreen user interface for such imaging systems.

Small, portable ultrasound imaging systems are available in the market today, including systems designated GE Logiq Book and Sonosite Titan. Mid-range ultrasound systems include the Philips Envisor. Both classes of ultrasound systems typically include a “hard” user interface (UI) consisting of physical keys in the form of a keyboard, buttons, slider potentiometers, knobs, switches, a trackball, etc. Most of these hard UI components are dedicated to specific control functions relating to use of the ultrasound system, and are labeled accordingly.

In addition, on some larger ultrasound systems, one or more electro-luminescent (EL) panel displays have been used to present a “soft” UI, typically consisting of variable, virtual keys on a touchscreen.

Both the hard and soft UI components are separate from the main display of the ultrasound system on which the generated ultrasound images are being displayed. The main display thus shows the ultrasound images and other textual or graphical information about the images, such as ECG trace, power level, etc., but does not allow direct user interaction, i.e., the user can only view the images being displayed but cannot interact with them via the main display. Rather, the user must turn to the hard UI components in order to change the parameters of the ultrasound images.

Some problems with existing ultrasound systems which comprise hard and soft UI components separate from the main display, e.g., a keyboard and an EL panel display, are added cost, complexity, power consumption, weight and maintenance of the separate components. It would therefore be desirable to incorporate both hard and soft UI components into the main display, thus eliminating the physical realizations of them and thereby avoiding the need to manufacture and maintain such separate UI components.

EP 1239396 describes a user interface for a medical imaging device with hard and soft components incorporated into a touchscreen display. The user interface includes a monitor on which an ultrasound image is displayed, a touchscreen in front of the monitor and activation areas and pop-up menus defined on the monitor screen. Each activation area is associated with a specific control function of the imaging system, e.g., mode select, penetration depth increase or decrease, zoom, brightness adjustment, contrast adjustment, etc., so that by touching the touchscreen over an activation area defined on the monitor screen, the associated function is performed.

US 2004/0138569 describes a graphical user interface for an ultrasound system in which a display screen has an image area and a separate control area on which control functions are defined, each in a separate area. The control functions are accessible via a touchscreen.

U.S. Pat. No. 6,575,908 describes an ultrasound system with a user interface which includes a hard UI component, i.e., a D-controller, and a touchscreen.

One problem with the prior art user interfaces is that they do not optimize the presentation of the activation areas. They also do not enable the manipulation of three-dimensional images.

It is an object of the present invention to provide a new and improved user interface for an ultrasound imaging system in which control functions are implemented as on-screen virtual devices.

It is another object of the present invention to provide a user interface for ultrasound imaging systems in which control functions are represented by activation areas on a touchscreen with an optimal presentation, namely, to facilitate the user's ability to easily select each activation area and/or to display activation areas simultaneous with ultrasound images while minimizing interference with the images and associated graphics.

In order to achieve these objects and others, a user interface for providing user control over device functions of an ultrasound imaging system in accordance with the invention includes a touchscreen on which ultrasound images are displayed and a plurality of activation areas selectively displayed on the touchscreen simultaneous with the display of ultrasound images. Each activation area has a unique assigned function relating to processing of the ultrasound images with an indication of the function being displayed on the activation area. A processor is coupled to the touchscreen for detecting a touch on the activation areas and performing the function associated with each activation area upon being touched. In this manner, all UI controls can be implemented as virtual controls by assigning the function of each control to an activation area so that the user can simply touch the activation area and effect the desired control. An assigned function can be a parameter relating to adjustment of the generation, processing or display of the ultrasound images, e.g., gain, compensation, depth, focus, zoom, or a display of additional activations areas, e.g., the display of pop-up menu which provide further available functions for selection.

One of the activation areas may be a segmented activation area including a plurality of activation areas arranged in a compact ring (or portion thereof) such that a center of each of these activation areas is equidistant from a common point, which might be the center of the segmented activation area. For example, in one embodiment, an activation area is defined on the touchscreen and when touched, causes the display of a pie menu of a plurality of additional activation areas. The pie menu is circular and each additional activation area has the form of a sector. The pie menu is centered at a location on the activation area touched by the user such that each of the additional activation areas is equidistant from the point of touch. This minimizes finger or stylus movement required by the user to select one of the additional activation areas. Instead of a circular pie menu, a polygonal menu can be displayed with each addition activational area having the shape of a trapezoid or triangle.

The function of each individual activation area can be to adjust a parameter in more than one direction, i.e., to increase or decrease gain, zoom, depth, etc., to thereby avoid the need to display two or more activation areas for a single parameter, e.g., one for gain increase and another for gain decrease. To obtain the adjustment of the parameter in the desired direction, the user sweeps across the activation area in the desired direction of the change in the form of a sliding touch, e.g., upward or downward, and the processor detects the sliding touch, determines its direction and then adjusts the parameter in the direction of the sliding touch. Such an activation area may have the form of a thumbwheel to provide the user with a recognizable control. A numerical readout can be displayed in association with the activation area to display a value of the parameter while the parameter is being adjusted. Moreover, the activation area or indication(s) within the activation area can change shape to conform to the shape drawn by the sliding touch.

In one embodiment, a profile of a parameter is adjustable by touching an activation area which responds to user touch by drawing a contour on the touchscreen in response to the track of the user's touch. The contour represents the control profile, i.e., a sequence of control values which vary according to the shape of the drawn contour. The control profile is used by the system to drive a control function that varies with some parameter such as time during a scan line. For example, the TGC (time-gain compensation) profile may be determined by a user-drawn TGC contour. The activation area is displayed with an initial, existing profile. Subsequent touches and drawing movements in the activation area by the user modify the profile, with the modified profile then being displayed for user review and possible further adjustment. The modifications may be strong, e.g., a single gesture replaces the existing contour, or they may be gradual, e.g., each gesture moves the profile to an intermediate position between the previous contour and the new one created by the gesture.

The activation areas can be provided with assigned functions which vary for different operation modes of the imaging system. The processor would thus assign functions relating to the imaging system to each activation area depending on an operation mode thereof. As the operation mode is changed, the functions of the activation areas, and their labels, shapes, colors, and degrees of transparency would change. For example, an activation area that acts as a button may indicate its function by means of its outline shape and a graphic displayed in the area, with no text label at all. Semi-transparency may be used to overlay activation areas upon each other or upon the underlying ultrasound image, so that display area consumption is minimized.

The user interface can also be designed to process handwritten text drawn or traced on the touchscreen by a finger, stylus or the like, using a handwriting recognition algorithm which converts touches on the touchscreen into text. By allowing for handwritten text entry, the user interface enables users to enter complex information such as patient data, comments, labels for regions of the images and the like.

An exemplifying ultrasound imaging system is capable of displaying real-time three-dimensional ultrasound images so that the activation areas have unique assigned functions relating to processing of three-dimensional images. The three-dimensional ultrasound images can be displayed as multiple planes oriented in their true spatial positions with respect to each other.

A method for providing user control over device functions of an ultrasound imaging system in accordance with the invention includes displaying ultrasound images on a touchscreen, defining a plurality of activation areas on a touchscreen simultaneous with the display of the ultrasound images, assigning a unique function relating to processing of the ultrasound images to each activation area, displaying an indication of the function on each activation area, positioning the activation areas to minimize interference with the simultaneous display of the ultrasound images, detecting when an activation area is touched, and performing the function associated with the touched activation area to change the displayed ultrasound images.

The appearance and disappearance of the activation areas may be controlled based on need for the functions assigned to the activation areas and/or based on activation by a user. This increases the time that the entire visual field of the touchscreen is occupied by the ultrasound images. In display formats where it is especially important to conserve space, activation areas with semi-transparent controls may be overlaid temporarily on other activation areas, and/or the image, and/or the informational graphics that accompany the image. Since the user's attention is focused on manipulating the controls and not on the fine detail of the underlying image and graphics, the semi-transparent controls do not diminish the utility of the display. The system changes made by the user's manipulation of a semi-transparent control may be visible through the control itself. For example, if the control is for image receive gain and its activation area is superimposed on the ultrasound image, the change in brightness of the image during manipulation of the control will be visible to the user not only from the region of the image surrounding the activation area, but underneath it as well, owing to the semi-transparency.

The activation areas may be arranged along a left or right side of a visual field of the touchscreen, or the top or bottom of the visual field, to minimize obscuring of the ultrasound images. The simultaneous display of the activation areas and ultrasound images enables the user to immediately view changes to the ultrasound images made by touching the activation areas.

The invention, together with further objects and advantages thereof, may best be understood by reference to the following description taken in conjunction with the accompanying drawings wherein like reference numerals identify like elements.

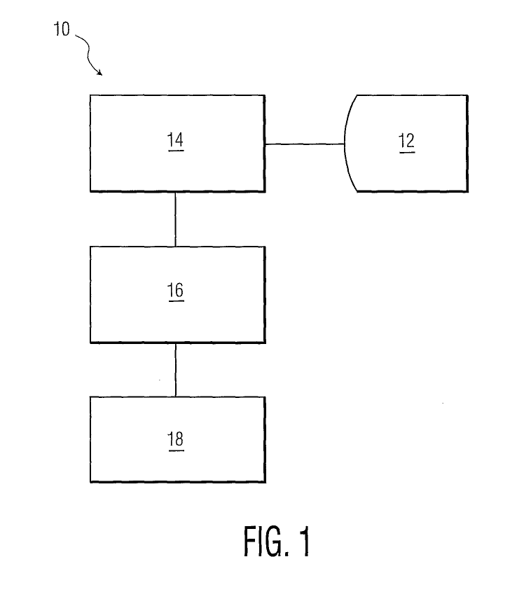

FIG. 1 is a block diagram of an ultrasound imaging system incorporating a user interface in accordance with the invention.

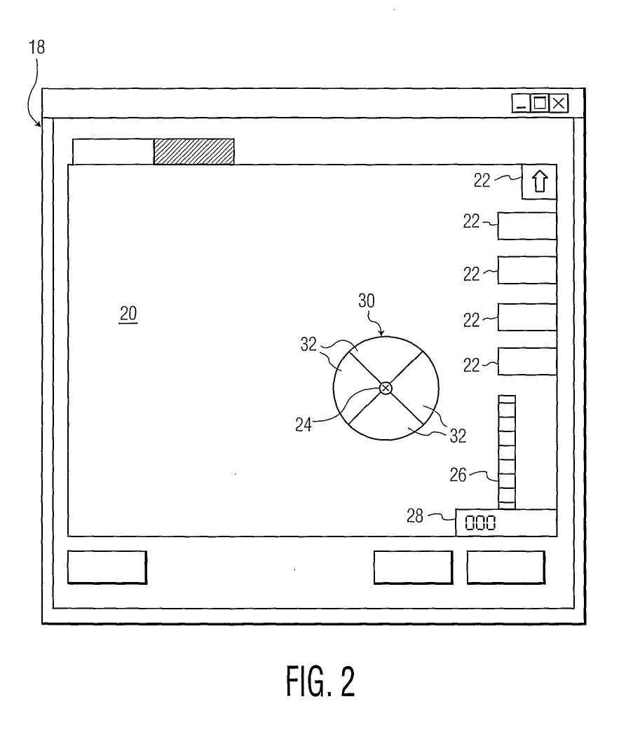

FIG. 2 shows a touchscreen of the ultrasound imaging system with a sample activation area layout.

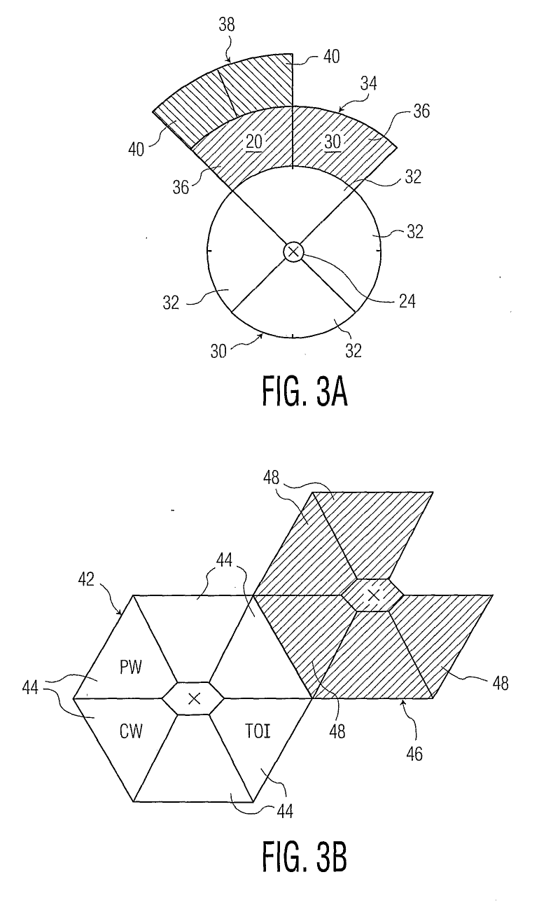

FIGS. 3A and 3B show two forms of cascading menus used in the user interface.

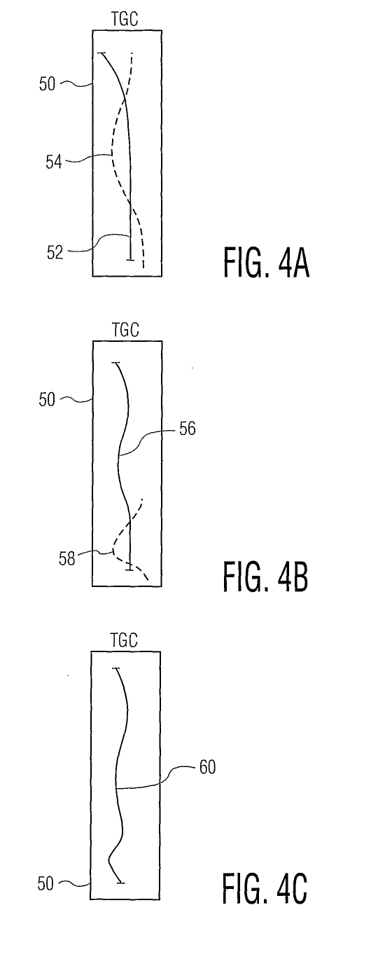

FIGS. 4A, 4B and 4C show an exemplifying activation area for a user-controllable value profile, and a sequence of operations to change the profile.

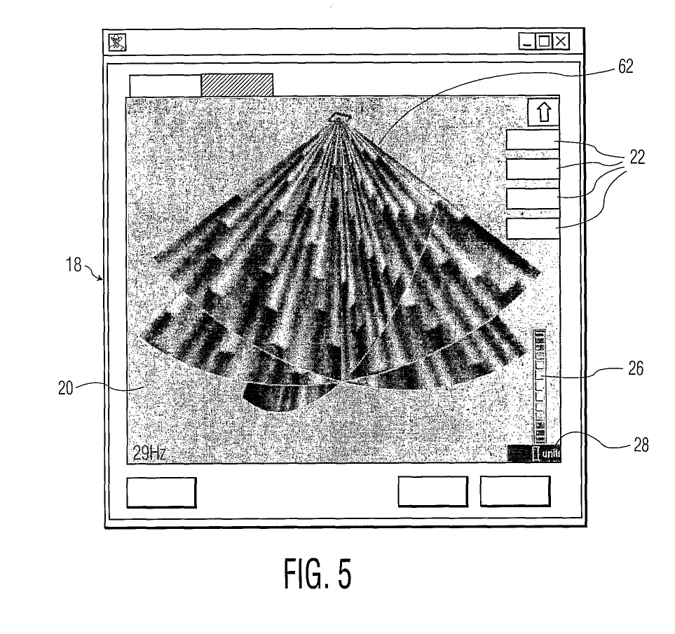

FIG. 5 shows a touchscreen of the ultrasound imaging system with a three-dimensional image and a sample activation area layout.

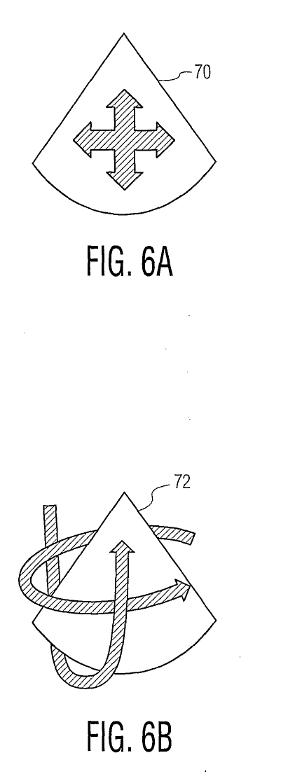

FIGS. 6A and 6B show exemplifying graphic symbols within activation areas for enabling the manipulation of the orientation of a displayed three-dimensional image.

Referring to FIG. 1, an ultrasound imaging system 10 in accordance with the invention includes an ultrasound scanner 12, an electromechanical subsystem 14 for controlling the ultrasound scanner 12, a processing unit or computer 16 for controlling the electromechanical subsystem 12 and a touchscreen 18 on which ultrasound images and virtual controls are displayed. The electromechanical subsystem 14 implements the electrical and mechanical subsystems of the ultrasound imaging system 10 apart from the computer software, monitor, and touchscreen interface. For example, the electromechanical subsystem 14 includes the necessary structure to operate and interface with the ultrasound scanner 12.

Computer 16 includes the necessary hardware and software to interface with and control the electromechanical subsystem 14, e.g., a microprocessor, a memory and interface cards. The memory stores software instructions that implement various functions of the ultrasound imaging system 10.

Touchscreen 18 may be implemented on a monitor wired to the computer 16 or on a portable display device wirelessly coupled to the computer 16, or both, and provides complete control over the ultrasound imaging system 10 by enabling the formation of command signals by the computer 16 indicative of desired control changes of the ultrasound imaging process. Touchscreen 18 may be a resistive, capacitive, or other touchscreen that provides an indication to the computer 16 that a user has touched the touchscreen 18, with his finger, a stylus or other suitable device, and a location of the touch. The location of the touch of the touchscreen 18 is associated with a specific control function by the computer 16, which control function is displayed at the touched location on the touchscreen 18, so that the computer 16 performs the associated control function, i.e., by generating command signals to control the electromechanical subsystem 14.

An important aspect of the invention is that input for controlling the ultrasound imaging system 10 is not required from hard UI components, for example, buttons, a trackball, function keys and TGC potentiometers and the like, nor from separate soft UI components, such as an EL (electro-luminescent) display. All of the control functions performed by such hard and soft UI components are now represented as virtual controls which are displayed on the touchscreen 18 along with the ultrasound images. The need for a separate keyboard for data entry, as well as the other hard UI components has therefore been eliminated.

FIG. 2 shows a sample of the layout of virtual controls on the touchscreen 18 during operation of the ultrasound imaging system 10. The touchscreen 18 displays in the available display area or visual field 20 either the ultrasound images in their entirety or the ultrasound images along with one or more superimposed activation areas 22, 24, 26 in a portion of the visual field 20. Activation areas 22, 24, 26 represent the usual controls of the ultrasound imaging system 10 which are implemented as on-screen virtual devices, including such hard UI controls as keys, buttons, trackball, and TGC potentiometers.

Computer 16 is programmable to allow the user to toggle between a full-screen display of the ultrasound images on the visual field 20 or a display of the ultrasound images and selected activation areas 22, 24, 26, which might depend on the imaging mode. When both ultrasound images and activation areas 22, 24, 26 share the visual field 20, computer 16 may be programmed to present a smaller, unobscured image with the activation areas 22, 24, 26 placed to one or more sides of the image, or alternatively to present a full size image with activation areas 22, 24, 26 superimposed on top of the image, optionally in a semi-transparent manner. These options may be configured by the user as preferences during system setup. Different imaging modes will result in the presentation of different activation areas 22, 24, 26 as well as different labels for the activation areas 22, 24, 26.

When the ultrasound images are displayed on the visual field 20 of the touchscreen 18 with the superimposed activation areas 22, 24, 26, the ultrasound images are displayed live so that control changes effected by touching the activation areas 22, 24, 26 are reflected immediately in the viewed images. Since the activation areas 22, 24, 26 are in the same visual field 20 as the images, the user does not have to shift his field of view from the image to separate UI components to effect a change, and vice versa in order to view the effects of the control change. User fatigue is thereby reduced.

The layout and segmenting of the activation areas 22, 24, 26 on the visual field 20 of the touchscreen 18 is designed to minimize interference with the simultaneous display of the ultrasound image and its associated graphics. Segmenting relates to, among other things, the placement of the activation areas 22, 24, 26 relative to each other and relative to the displayed ultrasound image, and the placement of further controls or portions of controls (e.g., addition activation areas 32, 36, 44 described below) when a particular one of the activation areas 22, 24 is in use. In particular, activation areas 22, 24, 26 appear in a segmented area of the visual field 20 when they are needed or when activated by the user (e.g., through the use of persistent controls which do not disappear). Preferably, the activation areas 22, 24, 26 are placed in a segmented area to a side of the image or on top of the image, e.g., using opaque (not semi-transparent) widget rendering. Alternatively, the image may be rendered large enough that it occupies at least a portion of the visual field 20 also occupied by activation areas 22, 24, 26. In that case, activation areas 22, 24, 26 may be rendered on top of the image, with optional semi-transparency as previously described. The activation areas 22, 24, 26 could be placed on the right side of the visual field 20 for right-handed users and on the left side for left-handed users. Right-handed or left-handed operation is a configurable option that may be selected by the user during system setup. Placement of the activation areas 22, 24, 26 on only one side of the visual field 20 reduces the possibility of the user's hands obscuring the image during control changes.

In one layout, activation areas 22, 24, 26 are set in predetermined positions and provided with variable labels and images according to the current imaging mode. The UI may be simplified so that only relevant or most recently used controls appear in the activation areas 22, 24, 26, but all pertinent controls can always be accessed by means of nested menus. The amount of nesting is minimized to reduce the number of required touches to perform any specific control function. The placement of nested menus constitutes further segmenting of the visual field 20 devoted to activation areas.

Each activation area 22 typically includes a label, mark, shape or small graphic image indicative of its function (e.g., a full word such as GAIN, FOCUS, DEPTH, or an abbreviation such as COMP, or a graphic denoting depth change) and when the user touches the touchscreen 18 at the location of a particular activation area 22, the computer 16 associates the touch with function and causes the ultrasound imaging system 10 to perform the associated function. The label on an activation area might be a function indicative of the display of a category of functions so that performing the associated function causes a pop-up menu of more specific functions to appear. For example, an activation area can be labeled as “GREYSCALE” and when touched causes additional activation areas to appear such as “DEPTH”, “SIZE”, etc. A mark can be arranged on activation areas which cause menus to appear, such as an arrow.

In some instances, it is necessary for the user to touch and sweep across the activation area 22 in order to indicate the exact function to be performed, i.e., a sliding touch. For example, the activation area 22 labeled GAIN is touched to both increase and decrease the gain and separate activation areas, one for gain increase and another for gain decrease, are not required. To increase gain, the user sweeps his finger one or more times in an upward direction over the activation area 22 labeled GAIN. Each upwardly directed sweep is detected and causes an increase in gain. On the other hand, to reduce the gain, the user sweeps his finger in a downward direction over the GAIN activation area.

Computer 16 can detect the sweeping over activation area 22 in order to determine the direction of the sliding touch by detecting individual touches on the touchscreen 18 and comparing the current touched location to the previous touched location. A progression of touched locations and comparison of each to the previous touched location provides a direction of the sliding touch.

Computer 16 is programmed to display a numerical readout 28 on the touchscreen 18 of the parameter the user is changing, as shown in FIG. 2. For example, when the GAIN activation area 22 is touched, readout 28 appears and the user can then adjust the gain by sweeping across activation area 26. However, once the user has stopped changing the gain, i.e., ceased sweeping across the activation area 26, the computer will cause the readout 28 and activation area 26 to disappear in order to maximize the area of the visual field 20 displaying the ultrasound images. The computer 16 thus controls the appearance and disappearance of activation areas 26 and readouts 28 of parameters the user is changing so that as large an area of the visual field 20 as possible is displaying the ultrasound images.

More particularly, to change a particular control value, the user may touch or otherwise activate the desired activation area 22 and then the “appearing” activation area 26. The activated area 22 may indicate it has been activated (to provide an indication as to what parameter is currently being adjusted) by changing its rendered state, such as with a highlight, light colored border outline, or the like. Readout 28 may then display the current (initial, pre-change) numerical value of the control function with the appropriate units. As the user makes changes to the control value via activation area 26, the readout 28 continuously updates and displays the current numerical value. Once the user has stopped changing the value of the control function, and a short period of time has elapsed since the last change, the readout 28 and activation area 26 may disappear to conserve display area available for displaying the image. Likewise, the activation area 22 returns to its un-selected, un-highlighted state.

In a similar manner, other settings such as FOCUS and DEPTH can be represented by a single activation area (see FIG. 2) yet enable changes in multiple directions by allowing the user to sweep his finger in a particular direction, e.g., upward/downward, or alternatively left/right (in the case of activation area 26 being rendered in a horizontal orientation), over the activation area 26 to obtain the desired directional change.

Although activation areas 22 are shown rectangular and spaced apart from one another, they can be any shape and size and placed adjacent one another. They may contain labels as shown in FIG. 2, or they may be graphical icons. They may employ colors to indicate their relation to other system functions or to indicate their activated state.

As shown in FIG. 2, activation area 26 has the appearance of a “hard” UI component, e.g., a thumbwheel. An advantage of activation area 26 appearing as a thumbwheel is that it provides a user-friendly feedback of the control parameter change to complement the numerical readout and/or change in the ultrasound image being displayed.

In a technique similar to that of activation area 26 appearing as a thumbwheel, a graphic representing a trackball may be displayed in the middle of an activation area that provides horizontal and vertical touch-and-drag input to system controls. Trackball controls are familiar to users of ultrasound system user interfaces, since most such systems in use today include a trackball for controlling parameters such as placement of a Doppler sample volume on the image, changing of image size or position, rotating the image, selecting amongst stored images, etc. Providing a trackball graphic and the corresponding control functions through an on-screen UI gives the user a migration path from a standard ultrasound scanner user interface with hard controls to the touchscreen UI of the invention.

Activation area 24 has a circular form and when touched, causes a pie-menu 30 to pop-up on the touchscreen 18 around it. Pie menu 30 provides an advantageous display of multiple activation areas 32 occupying substantially the entire interior of a circle, each activation area 32 being a slice or arcuate segment of the circle, i.e. a sector or a portion of a sector. Activation area 24 can include a general label or mark indicative of the control functions associated with activation areas 32 so that the user will know which activation areas 32 will appear when activation area 24 is touched. After pie menu 30 pops up, activation area 24 at the center of the pie is replaced with an “X” graphic, indicating that touching it will cause the pie menu to be removed, canceling the system change. Upon further selection of an activation area 32 within the pie menu 30, the activation area 24 at the center of the pie menu 30 may be replaced by a “check” graphic to indicate that it may be used to confirm the selection(s) and cause computer 16 to remove the pie menu 30.

Pie menus 30 provide the user with the ability to select one of a plurality of different control functions, each represented by one of the activation areas 32, in a compact and efficient manner. The possible control functions are very closely packed in the pie shape, but do not overlap and thereby prevent erroneous and spurious selection of an activation area 32. Also, the computer 16 is programmed to cause the pie menu 30 to appear with its center at the location on the activation area 24 touched by the user. In this manner, the pie menu 30 will pop-up in a position in which the activation areas 32 are all equidistant from the position of the finger when it caused the pie menu 30 to pop up on-screen, i.e., the centers of the activation areas 32 are equidistant from a common point on the touchscreen, namely the center of the activation area 24. Rapid selection of any activation area 32 is achieved, mitigating the time penalty associated with having to invoke the menu from its hidden state as well as reducing finger or stylus movement to arrive at the desired activation area 32.

If the pie menu 30 appears on the visual field 20 for a period of time without a touch of any of the activation areas 32 being detected by the computer 16, the computer 16 can be programmed to cause the pie menu 30 to disappear in order to maximize the area of the visual field displaying the ultrasound images.

Instead of pie menu 30 being circular and having four substantially identical activation areas 32 with each extending over a 90° segment as shown, it can also have a slightly oval shape and include any number of activation areas, possibly extending over different angular segments.

Cascading pie menus can also be provided whereby from activation area 24, a single pop-up pie menu 30 will appear with multiple activation areas 32 and by touching one of the activation areas 32, another pop-up pie menu will appear having the same circular shape as pie menu 30 or a different shape and form.

For example, referring to FIG. 3A, pie menu 30 has four activation areas 32 shaped as equally spaced sector segments. Touching any one of the activation areas 32 causes a cascaded menu to appear in an extended portion of the respective sector. If the “Grayscale” activation area is touched, for instance, the cascaded menu 34 appears, containing in this case two activation areas 36 which are preferably spaced equidistant from the center point of pie menu 30. Similarly, if activation area 36 labeled “2D” is subsequently touched, another cascaded menu 38 appears, again with two activation areas 40, extending from the activation area 36 labeled “2D”. Activation areas 40 are preferably spaced equidistant from the center point of pie menu 30. Although this example shows a particular number and pattern of activation areas 32, 36, 40 in cascaded menus 30, 34, 38 (four, then two, then two), it will be understood by those skilled in the art that any number of cascades and any number of segments within each cascade level could be implemented, subject to the constraints of limited display area and minimum font size for the labels. Although labels for the activation areas 32, 36, 40 are shown in this example, other indicators of function could be used instead, such as graphic images, colors, or shapes. After touching the desired activation area(s) 32, 36, 40 in one or more cascaded menu 30, 34, 38, the user may confirm the final choice of activation area 32, 36, 40, and thereby the system function desired, by any of various means including but not limited to waiting for a predetermined “quiet” period to expire with no further selections, or by double-touching (i.e., quickly touching twice) the desired activation area, or by touching the center of the pie menu 30 at activation area 24, where the graphic displayed therein may have been changed by computer 16 after the first selection of an activation area 32, replacing the initially displayed “X” graphic offering cancellation of the selection to a “check” graphic offering confirmation of the final selection.

Alternatively, other types of cascading, segmented activation areas or pop-up menus can appear. For example, referring now to FIG. 3B, a pie menu 42 with trapezoidal activation areas 44 can be used, enabling the formation of a cascade submenu 46 defining a set of segmented polygons constituting activation areas 48. The center points of the activation areas 44, 48 may be possibly equidistant from a common point on the touchscreen. In each cascaded submenu 46, one of the polygons 48 abuts the selected activation area 44 in the parent pie menu 42. Preferably, this abutting polygon 48 contains the dominant choice in the cascaded submenu 46. In FIG. 3B, the cascaded submenu 46 for the “Flow” activation area of the parent pie menu 42 is displayed. The dominant choice on the cascaded submenu 46 is “Gain”, and its activation area 48 abuts the “Flow” activation area, because selecting “Gain” after selecting “Flow” will result in the least movement and effort for the user.

Turning now to FIGS. 4A, 4B and 4C, an activation area 50 representing a series of control values is exemplified. Activation area 50, as shown in this example, controls the ultrasound TGC function, and consists of an elongated rectangle with a border drawn to define the region in which the user's touch will have an effect on the TGC control profile. The activation area 50 is first displayed, preferably, by means of touching another activation area 22 labeled “TGC”. The existing TGC profile is initially graphed in the activation area 50, using profile curve 52 as shown in FIG. 4A (the solid line). The profile curve 52 represents the relative amount of receive gain along the ultrasound scan lines in the image as a function of scan depth, where the starting scan depth is at the top of the profile and deeper depths are lower on the profile. Where the profile 52 bends to the right hand side of the activation area 50, the relative gain in the scan lines is greater. Thus, minimum gain is at the left side of the activation area 50. This arrangement matches the typical layout of hard TGC controls on a conventional ultrasound scanning system.

The user may change the TGC profile by touching continuously in the activation area 50 and drawing a new touch path 54 with a finger, stylus or the like. In this example, the TGC control preferably changes gradually in response to repetitions of touch path 54. An exemplary sequence of two touch paths 54, 58 are shown in FIGS. 4A-4C. In FIG. 4A, the touch path 54 decreases gain around the midfield depth, as indicated by the leftward bend of the path around the middle of activation area 50. The response of the system is shown in FIG. 4B, where computer 16 has redrawn the profile curve in response to the touch path 54 shown in FIG. 4A. The revised TGC profile 56 has a bend to the left around the mid-field, but not as distinct and extensive as the touch path 54, reflecting the gradual, averaging algorithm used to make changes to the profile. An exemplifying algorithm averages the values collected from the touch path 54 with the values stored in the previous TGC profile curve 52. This averaging facilitates the user's ability to see the changes he is making without obscuring them with his finger, and also allows the user to make fine changes by repeated gestures (touch paths) within the small, narrow activation area 50. Both of these advantages suit the needs of the compact visual field 20.

In this example, and referring to FIG. 4B, the user then draws a second touch path 58, which adjusts the TGC profile only near the deepest depth, with a relatively short touch path. The user begins touch path 58 near the bottom of the activation area 50. The computer 16 therefore makes no change to TGC profile curve 56 in the shallower depths. FIG. 4C shows the resulting TGC profile curve 60, accumulating changes from both preceding touch paths 54, 58. If the user is satisfied with the TGC profile shape, he leaves the activation area 50 untouched for a short quiet time (typically turning to some other task), and computer 16 automatically removes the activation area 50 from the visual field 20.

Using activation areas 22, 24, 26 and the described variations thereof, all of the possible control functions of the ultrasound system 10 can be implemented as virtual controls on the touchscreen 18.

The ultrasound system 10 described above can be combined with a display of real-time three-dimensional ultrasound images wherein the images are rendered as either semi-transparent volumes or as multiple planes oriented in their true spatial positions with respect to each other. The latter image format is exemplified by the test pattern 62 of three superimposed images planes shown in the center of the visual field 20 on the touchscreen 18 in FIG. 5. Touchscreen 18 allows manipulation of specific three-dimensional parameters, such as the orientation of the image, the degree of opacity, etc., via the activation areas 22 which are labeled with control functions specific to three-dimensional images. Activation areas 22 are in the upper right hand corner while the frame rate is displayed in the lower left hand corner.

For example, an activation area 22 may contain a graphic symbol indicating horizontal/vertical translation of the image, as exemplified by graphic 70 in FIG. 6A. When this activation area is touched, it preferably changes to a highlighted state, e.g., by means of a highlighted border or a change in graphic color, and the user may then translate the image horizontally or vertically on the visual field 20 by touching anywhere on the image and dragging. After a short period of no image movement by the user, or if a different activation area is touched, the activation area 22 associated with image translation is automatically un-highlighted by computer 16 and the translation function is disabled. As a further example, an activation area 22 may contain a graphic symbol for image rotation, as illustrated by graphic 72 in FIG. 6B. When this activation area is touched, it preferably changes to a highlighted state, and the user may then rotate the 3D image about a horizontal or vertical axis in the visual field 20 by touching anywhere on the image and dragging. After a short period of no image rotation by the user, or if a different activation area is touched, the activation area 22 associated with image rotation is automatically un-highlighted by computer 16 and the rotation function is disabled.

In addition to touchscreen input, the same system display would also allow user input via stylus or other suitable device. So-called dual-mode screens are available today on “ruggedized” tablet PCs. The stylus input would be useful for entering high resolution data, such as patient information via a virtual keyboard or finely drawn region-of-interest curves for ultrasound analysis packages.

The user interface can also be designed to process handwritten text drawn or traced on the touchscreen by a finger, stylus or the like. To this end, the user interface would include a handwriting recognition algorithm which converts touches on the touchscreen into text and might be activated by the user touching a specific activation area to indicate to the user interface that text is being entered, e.g., an activation area 22 designated “text”, with the user being able to write anywhere on the touchscreen. Alternatively, a specific area of the touchscreen might be designated for text entry so that any touches in that area are assumed to be text entry. By allowing for handwritten text entry, the user interface enables users to enter complex information such as patient data, comments, labels for regions of the images and the like. This information would be stored in association with the ultrasound images from the patient.

The touchscreen user interface described above is particularly suited for small, portable ultrasound systems where cost and space are at a premium. Thus, tablet PCs are ideal applications for the user interface.

Moreover, ultrasound scanners are becoming very small so that in one implementation of the invention, an ultrasound imaging system includes an ultrasound scanning probe with a standard interface connection (wired or wireless) and integrated beamforming capabilities, a tablet PC with an interface connection to the scanning probe and the user interface described above embodied as software in the tablet PC and with the ability to form the activation areas and display the ultrasound images on the screen of the tablet PC.

Although the user interface in accordance with the invention is described for use in an ultrasound imaging system, the same or a similar user interface incorporating the various aspects of the invention can also be used in other types of medical diagnostic imaging systems, such as an MRI system, an X-ray system, an electron microscope, a heart monitor system, and the like. The options presented on and selectable by the virtual controls would be tailored for each different type of imaging system.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1. In an ultrasound imaging system, a user interface for providing user control over device functions of the imaging system, comprising:
a touchscreen (18);
a segmented activation area (30, 42) defined on said touchscreen (18), said segmented activation area (30,42) including a plurality of activation areas (32, 44) wherein each of said plurality of activation areas (32, 44) has a unique assigned function relating to the imaging system with an indication of said function being displayed on said activation area (32, 44); and
a processor (16) coupled to said touchscreen (18) for detecting a touch on said activation areas (32, 44) defined on said touchscreen (18) and performing the function associated with each of said activation areas (32, 44) upon being touched.

2. The user interface of claim 1, wherein said plurality of activation areas (32, 44) are arranged relative to one another such that center points of said plurality of activation areas (32, 44) are equidistant from a common point on said touchscreen (18), said plurality of activation areas (32, 44) being arranged in a ring around said common point.

3. The user interface of claim 1, wherein said segmented activation area (30) is circular and each of said plurality of activation areas (32) has a form of at least a portion of a sector, and said plurality of activation areas (32) occupy substantially the entire space of said segmented activation area (30).

4. The user interface of claim 1, wherein said segmented activation area (42) is a polygon and each of said plurality of activation areas (44) has a form of at least a portion of a polygon, and said plurality of activation areas (44) occupy substantially the entire space of said segmented activation area (42).

5. The user interface of claim 1, wherein the function associated with at least one of said plurality of activation areas (32, 44) is display of a submenu (34, 46) of a plurality of additional activation areas (36, 38, 48), each of said additional activation areas (36, 48) having the form of a portion of a sector and a unique assigned function relating to the imaging system with an indication of said function being displayed on said additional activation area (36, 48).

6. The user interface of claim 5, wherein said segmented activation area (30) is substantially circular, said additional activation areas (36, 38) being arranged adjacent to an outer surface of said at least one of said plurality of activation areas (32) such that said additional activation areas (36, 38) have center points equidistant from a center of said segmented activation area (30).

7. The user interface of claim 5, wherein said segmented activation area (42) is polygonal, said additional activation areas (48) being arranged around a common point such that said additional activation areas (48) have center points equidistant from said common point and one of said additional activation areas (48) is adjacent to an outer surface of said at least one of said plurality of activation areas (44).

8. The user interface of claim 1, further comprising an additional activation area (24) defined on said touchscreen (18) which when touched, causes said segmented activation area (30) to appear on said touchscreen (18) with its center at the touched location on said additional activation area (24), said segmented activation area (30) being related to said additional activation area (24).

9. In an ultrasound imaging system, a user interface for providing user control over device functions of the imaging system, comprising:
a touchscreen (18);
a first activation area (24) defined on said touchscreen (18) which when touched, causes a plurality of related second activation areas (32) to appear on said touchscreen (18), each of said second activation areas (32) having a unique assigned function relating to the imaging system with an indication of said function being displayed on said second activation area (32); and
a processor (16) coupled to said touchscreen (18) for detecting a touch on said first and second activation areas (24, 32) defined on said touchscreen (18) and performing the function associated with each of said first and second activation areas (24, 32) upon being touched.

10. The user interface of claim 9, wherein said second activation areas (32) are arranged in a single segmented activation area (30).

11. The user interface of claim 9, wherein said second activation areas (32) comprise an activation area (26) having the form of a thumbwheel for adjusting a function value and an activation area (28) providing a readout of the function value.

12. In an ultrasound imaging system, a user interface for providing user control over device functions of the imaging system, comprising:
a touchscreen (18);
an activation area (22, 26, 40) defined on said touchscreen (18), said activation area (22, 26, 40) having an assigned parameter or profile of a parameter relating to the imaging system with an indication of said parameter or profile being displayed on said activation area (22, 26, 40); and
a processor (16) coupled to said touchscreen (18) for detecting a sliding touch over said activation area (22, 26, 40) and adjusting the parameter or profile based on the sliding touch.

13. The user interface of claim 12, wherein said activation area (26) has the appearance of a thumbwheel for adjusting the assigned parameter and said processor (16) is arranged to detect a direction of the sliding touch over said activation area (26).

14. The user interface of claim 13, further comprising a numerical readout (28) arranged in association with said activation area (26) to display a value of the assigned parameter.

15. The user interface of claim 12, wherein said processor (16) is arranged to display an initial profile of the parameter, adjust the assigned profile based on the sliding touch, and display the adjusted profile.

16. An ultrasound imaging system (10), comprising:
an ultrasound scanner (12);
a touchscreen (18);
a processor (16) coupled to said ultrasound scanner and said touchscreen (18) and arranged to display real-time three-dimensional ultrasound images on said touchscreen (18); and
a plurality of activation areas (22, 26) defined on said touchscreen (18), each of the activation areas (22, 26) having a unique assigned function relating to processing of a three-dimensional image with an indication of said function being displayed on said activation area (22, 26), said processor (16) being arranged to detect touches of said activation areas (22, 26) and perform the function associated with each of said activation areas (22, 26) upon being touched.

17. The system of claim 16, wherein said processor (16) is arranged to display the three-dimensional ultrasound images as multiple planes oriented in their true spatial positions with respect to each other.

18. The system of claim 16, wherein one of said activation areas is arranged to enable vertical/horizontal translation of the displayed ultrasound images.

19. The system of claim 16, wherein one of said activation areas is arranged to enable rotation of the displayed ultrasound images.

20. In an ultrasound imaging system, a user interface for providing user control over device functions of the imaging system, comprising:
a touchscreen (18);
a plurality of activation areas (22) defined on said touchscreen (18); and
a processor coupled to said touchscreen (18) for assigning unique functions relating to the imaging system to each of said activation areas (22) depending on an operation mode of the imaging system such that each of said activation areas (22) has a variably assigned function, an indication of said function being displayed on said activation area (22), said processor (16) detecting a touch on said activation areas (22) defined on said touchscreen (18) and performing the function associated with each of said activation areas (22) upon being touched.

21. A method for providing user control over device functions of an ultrasound imaging system, comprising:
displaying ultrasound images on a touchscreen (18);
defining a plurality of activation areas (22, 24, 26) on a touchscreen (18) simultaneous with the display of the ultrasound images, each of the activation areas (22, 24, 26) having a unique assigned function relating to processing of the ultrasound images with an indication of the function being displayed on the activation area (22, 24, 26);
positioning the activation areas (22, 24, 26) to minimize interference with the simultaneous display of the ultrasound images;
detecting when one of the activation areas (22, 24, 26) is touched; and
performing the function associated with the touched activation area (22, 24, 26) to change the displayed ultrasound images.

22. The method of claim 21, further comprising controlling the appearance and disappearance of activation areas (22, 24, 26) based on need for the functions assigned to the activation areas (22, 24, 26) or based on activation by a user.

23. The method of claim 21, wherein the positioning step comprises arranging all of the activation areas (22, 24, 26) along a left or right side of a visual field (20) of the touchscreen (18).

24. The method of claim 21, further comprising assigning variable functions and indications to the activation areas (22, 24, 26) depending on an operation mode of the imaging system.

25. The method of claim 21, wherein the defining step includes defining at least one of the activation areas as a segmented activation area (30) including a plurality of distinct activation areas (32) each having the form of at least a portion of a sector and a unique assigned function relating to the imaging system with an indication of the function being displayed on the activation area (32).

26. The method of claim 21, wherein the function assigned to one of the activation areas (24) is display of a submenu (30) of a plurality of additional activation areas (32), further comprising centering display of the submenu (30) at a location on the activation area (24) touched by the user such that each of the additional activation areas (32) is equidistant from the point of touch.

27. The method of claim 21, wherein the function assigned to at least one of the activation areas (22, 26, 50) is to provide adjustment of a parameter or profile of a parameter in multiple directions, further comprising detecting a sliding touch over the at least one activation area (22, 26, 50) and adjusting the parameter based on the sliding touch.

28. The method of claim 21, wherein real-time three-dimensional ultrasound images are displayed, the activation areas (22) being assigned functions relating to processing of three-dimensional images.

29. The method of claim 21, wherein the function assigned to at least one of the activation areas (26) is to provide adjustment of a parameter, further comprising displaying a numerical readout (28) of the parameter while the at least one activation area (26) is being touched and removing the numerical readout (28) from the touchscreen (18) once touching of the at least one activation area (26) has ceased.

30. The method of claim 21, further comprising selectively switching a visual field (20) of the touchscreen (18) from a first mode in which the entire field of view is occupied by the ultrasound images to a second mode in which the activation areas (22, 24, 26, 32) are displayed in the field of view.

31. The method of claim 21, further comprising the step of displaying the activation areas (22, 24, 26, 32) in a semi-transparent manner over the displayed ultrasound images.

32. The method of claim 21, further comprising defining an activation area for text entry and converting text handwritten in the activation area into data for storage in association with the ultrasound images.
